(19) United States
(12) Patent Application Publication
(10) Pub. No.: US 2005/0089490 A1
(43) Pub. Date: Apr. 28, 2005

(54) Hair gels based on polymers having a low glass transition temperature

(75) Inventors: Janusz Jachowicz, Bethel, CT (US); Jui-Chang Chuang, Wayne, NJ (US); Roger L. McMullen JR., Bloomfield, NJ (US); Susan Chen, Fort Lee, NJ (US)

Correspondence Address: INTERNATIONAL SPECIALTY PRODUCTS;Attn: William J. Davis, Esq., Legal Department, Building No. 8, 1361 Alps Road, Wayne, NJ, 07470-3529, US

(73) Assignee: ISP INVESTMENTS INC.

(21) Appl. No.: 10/950,924

(22) Filed: Sep. 27, 2004

Related U.S. Application Data
(60) Provisional application No. 60/514,961, filed on Oct. 28, 2003.

(51) Int. Cl. A61K 7/06; A61K 7/11

(52) U.S. Cl. 424/70.11; 424/70.16; 424/70.17

(57) ABSTRACT

What is described herein is a hair care composition including a water soluble or water dispersible styling polymer characterized by a glass transition temperature below room temperature, and a gel-forming polymer which has a glass transition temperature higher than or lower than room temperature.

### CROSS-REFERENCE TO RELATED PATENT APPLICATIONS

This application is based upon Provisional Application Ser. No. 60/514,961, filed Oct. 28, 2003.

### BACKGROUND OF THE INVENTION

1. Field of the Invention

This invention relates to a hair care compositions, and, more particularly to hair gels based on polymers having a low glass transition temperature.

2. Description of the Prior Art

Hair gels typically contain a styling polymer such as, polyvinylpyrrolidone (PVP), polyvinylpyrrolidone-vinyl acetate copolymer (PVP/VA), or Polyquaternium-11; a gel forming polymer such as Carbomer®, Aculyn®28, hydroxyethylcellulose or Stabileze®, and other additive ingredients such as neutralizers, protectants, plasticizers, etc. Styling polymers based on acrylate or vinyl pyrrolidone chemistry are usually characterized by a relatively high glass transition temperature, T_{g}. For example, the T_{g} of PVP ranges from 150° C. to 170° C. depending on its molecular weight. The T_{g} of a PVP/VA copolymer depends on the monomer composition but usually ranges from 55° C. to 109° C. Gel-forming polymers such as Carbomer® or Stabileze® possess a glass transition of 100° C. and 120° C., respectively. Thus, the film formed from such hair gel products are typically characterized by a glass transition temperature in the range of from 100° to 200° C. Accordingly, such films are usually perceived by the consumer as being stiff, brittle and non-flexible.

Accordingly, another object of this invention is to formulate hair gels characterized by low to moderate stiffness and a natural and flexible feel.

Another object of this invention to provide hair gels based on polymers which have a low glass transition temperature.

### SUMMARY OF THE INVENTION

What is described herein are hair gel compositions which include styling polymers characterized by glass transition temperatures below room temperature. Such hair styling formulations produce flexible styling films. The low T_{g} polymer is blended herein in appropriate proportions with gel-forming polymers so that the resultant combination has a predetermined, relatively low glass transition temperature. Such hair gels are capable of imparting a flexible feel to hair.

The composition of the polymer blends of the invention suitably comprise about 1-4 wt. % of the styling polymer and about 0.3-1 wt. % of the gelling polymer, most preferably, about 2% and 0.5%, respectively.

### DETAILED DESCRIPTION OF THE INVENTION

In order to formulate hair gels characterized by low to moderate stiffness with a natural and flexible feel, the styling polymer of this invention is selected from polyurethanes, polyacrylates, polyolefins and polyesters, particularly polymers having a T_{g} lower than room temperature. Such polymers are present in the rubbery state and are characterized by relatively low modulae and very high values of extension at break. These polymer materials impart a flexible and natural feel to hair, similar to that of untreated hair. It was found, surprisingly, that deposition of such polymers on the surface of hair does not result in detrimental modification of hair friction, i.e. no increase in friction or drag. In addition, it was found that these polymers do not need to be water-soluble. Accordingly, water-insoluble polyurethanes and/or polyacrylates can be formulated into hair gel products which can be easily applied to hair and impart good tactile properties thereto. Furthermore, they are easily washed off from hair by shampooing.

In this invention, the low glass transition styling polymers suitably are co-formulated with a low or high glass transition temperature gel-forming polymer to form a blend of polymers having a relatively low glass transition temperature. Such blends are flexible film formers because the styling polymer is typically used in large excess. For example, for a hair gel composition containing 2% of a polyacrylate having a low T_{g} and 0.5% Carbomer® having a glass transition temperature of about 0° C., the resulting homogenous polymer blend has a glass transition temperature of about 15° C. When this polymer blend is non-homogenous with an excess of the styling polymer therein, a continuous phase is formed which is characterized by the low glass transition temperature polymer with embedded microdomains of the gel-forming polymer.

For example, a blend of a high glass transition polymer such as polyvinylpyrrolidone (PVP) and a low glass transition gel-forming polymer, e.g. Aculyn®28, provides products with acceptable flexibility. However, even more flexible systems can be obtained by blending a low glass transition styling polymer with a low glass transition gelling polymer. These compositions demonstrate both high flexibility and high plasticity. The mechanical and tactile properties of such polymer gels also can be varied advantageously by including low molecular weight additives such as plasticizers in the composition.

Flexible compositions based on blends of a low glass transition styling polymer and a second polymer can be employed for preparing many different types of cosmetic compositions such as hair mousses, hair lotions, hair tonics, hair spray gels, hair waxes, etc. The function of the second polymer is to provide an initial gluing effect after application of the product on hair.

A particularly useful application for the polymer blend herein is a hair styling gel. Other hair styling products, and leave-in hair conditioners also have similar film formation capabilities. Accordingly, the composition of the invention can be used in many hair care formulations including hair conditioning products, hair body-building products, and the like.

The invention will now be described with particular reference to the preparation of flexible styling products containing (a) a water soluble or water dispersible styling polymer characterized by a glass transition temperature below room temperature, and (b) a gel-forming polymer characterized by a glass transition temperature higher than or lower than room temperature, and optionally, (c) other typical hair formulation ingredients such as neutralizing agents, conditioning agents, protectants, and the like.

The composition of the polymer blends of the invention suitably comprise about 1-4 wt. % of the styling polymer and about 0.3-1 wt. % of the gelling polymer, most preferably, about 2% and 0.5%, respectively.

### EXAMPLE 1

### Carbopol® 940 Slurry

| Ingredient | % (W/W) | Mass (g) |
|---|---|---|
| Carbopol ® 940 (Noveon) | 2% | 10 |
| Deionized H₂O | 98% | 490 |

Dispersed Carbopol® 940 into water and mixed until air bubbles were released and the slurry became homogenous (translucent, off-white).

### EXAMPLE 2

### Hair Gel Based on a Water Dispersible Polyurethane Rubber

| | | |
|---|---|---|
| Ingredient | % (W/W) | Mass (g) |

Phase A

| | | |
|---|---|---|
| Carbopol ® 940 slurry (Ex. 1) | 35% | 175 |
| Deionized H₂O | 49% | 246 |
| Phase B | | |
| Bayhydrol ® PR 240 (Bayer, 40% soln.) | 5% | 25 |
| Deionized H₂O | 10% | 50 |
| Phase C | | |
| AMP-95 (Angus, 95% soln.) | 1% | 4 |

The composition had a pH of approximately 7.

### EXAMPLE 3

### Hair Gel Based on a Water Dispersible Polyurethane Rubber

| Ingredient | % (W/W) | Mass (g) |
|---|---|---|
| Phase A | | |
| Carbopol ® 940 slurry (Ex. 1) | 35% | 175 |
| Deionized H₂O | 49% | 246 |
| Phase B | | |
| Bayhydrol ® DLN (Bayer, 39% soln.) | 5% | 25.64 |
| Deionized H₂O | 10% | 50 |
| Phase C | | |
| AMP-95 (Angus, 95% soln.) | 1% | 3.36 |

The composition had a pH of about 7.

### EXAMPLE 4

### Hair Gel Based on a Water-Soluble Polyester Having a Glass Transition Temperature Below Room Temperature

| Ingredient | % (W/W) | Mass (g) |
|---|---|---|
| Phase A | | |
| Carbopol ® 940 Slurry | 35% | 87.5 |
| Deionized H₂O | 44% | 110.5 |
| Phase B | | |
| Polyester (10% soln.) | 2.0% | 50 |
| Phase C | | |
| AMP-95 (Angus, 95% soln.) | 1% | 2 |

### EXAMPLE 5

### Hair Gel Based on a Water Dispersible Polyester Rubber

| Ingredient | % (W/W) | Mass (g) |
|---|---|---|
| Phase A | | |
| Carbopol ® 940 slurry (Ex. 1) | 35% | 87.5 |
| Deionized H₂O | 44% | 110.5 |
| Phase B | | |
| Polyester (10% soln.) | 2.0% | 50 |
| Phase C | | |
| AMP-95 (Angus, 95% soln.) | 1% | 2 |

### EXAMPLE 6

### Hair Gel Based on an Anionic Associative Thickener and a Water Dispersible Polyurethane Rubber

| | | |
|---|---|---|
| Ingredient | % (W/W) | Mass (g) |

Phase A

| | | |
|---|---|---|
| Aculyn ® 28 (Rohm &Haas) (20.8% active) | 0.7% | 3.4 |
| Phase B | | |
| Bayhydrol ® PR 240 (Bayer, 40% soln.) | 2% | 5 |
| DI Water | q.s. | 91.1 |
| Phase C | | |
| AMP-95 (Angus, 95%) | | 0.5 |

Phases A and B were combined. Then Phase C was added dropwise with mixing until homogenous.

### EXAMPLE 7

### Hair Gel Based on Carbomer® 940 and Acrylate

| Ingredient | % (W/W) | Mass (g) |
|---|---|---|
| Phase A | | |
| Carbopol ® 940 slurry | 0.5 | 25 |
| Phase B | | |
| Acrylate | 2.0 | 9.5 |
| DI Water | q.s. | 65.5 |
| AMP | q.s. to pH 7 | |

### EXAMPLE 8

### Hair Gel Based on Carbomer® 940 and PVP K-30

| | | |
|---|---|---|
| Ingredient | % (W/W) | Mass (g) |

Phase A

| | | |
|---|---|---|
| Carbomer ® 940 (Noveon) (2% slurry) | 0.5 | 25 |
| Phase B | | |
| PVP K-30 | 2.0 | 2 |
| DI Water | | 73 |
| Phase C | | |
| AMP | q.s. to pH 7 | |

### EXAMPLE 9

### Hair Gel Based on Aculyn® 28 and PVP K-30

| Ingredient | % (W/W) | Mass (g) |
|---|---|---|
| Phase A | | |
| Aculyn ® 28 (R&H) | 0.5 | 2.50 |
| Phase B | | |
| PVP K-30 | 2.0 | 2.0 |
| DI Water | | 95.5 |
| Phase C | | |
| AMP | q.s. to pH 7.5 | |

Dissolved PVP K-30 in water; added Phase A; then added Phase C dropwise with mixing until the product became homogenous.

### EXAMPLE 10

### Evaluation of Polyurethane Dispersions in Solutions Applied to Hair

Hair loops were prepared as described previously [JSCC, 47, 73 (1996)]. Then 1% and 3% solutions of Bayhydrol® PR 240 and Bayhydrol® DLN were prepared by diluting the commercial concentrates with deionized water. The solutions were applied to hair in an amount of 150 mg per hair tress. The treated hair was allowed to dry and was tested for flexibility by using a Texture Analyzer (DHSA traces) [JCS, 53, 345 (2002)].

The results demonstrated that the polymers produced a flexible modification of hair as evidenced by high E10/E1 values. Also, the stiffness ratio value for such treatment was relatively low, varying from 2 to 6.5.

### EXAMPLE 11

### Evaluation of Polyester Solutions Applied to Hair

Similar DHSA traces were obtained for hair treated with 5.71% solutions of a polyester prepared by dissolving appropriate amounts of the solid polymer in water. The data demonstrated flexibility and plasticity of polyesters.

### EXAMPLE 12

### DHSA Evaluation of Hair Gels Prepared According to Examples 1-9

Similar DHSA traces were obtained for hair treated with gels prepared in Examples 1-7 and 9. The data demonstrated improved flexibility and plasticity as compared to the gel of Example 8.

While the invention has been described with particular reference to certain embodiments thereof, it will be understood that changes and modifications may be made which are within the skill of the art. Accordingly, it is intended to be bound only by the following claims, in which:

1. A hair care composition comprising
(a) a water soluble or water dispersible styling polymer characterized by a glass transition temperature below room temperature, and
(b) a gel-forming polymer characterized by a glass transition temperature higher than or lower than room temperature.

2. A hair care composition according to claim 1 wherein (a) is selected from a polyurethane, a polyacrylate, a polyolefin, or a polyester.

3. A hair care composition according to claim 1 wherein (a) is present in an amount of 1-4 wt. %; and (b) in an amount of 0.3-1%.

4. A hair care composition according to claim 1 wherein (a) is about 2%, and (b) is about 0.5%.

5. A hair care composition according to claim 1 which is a gel, mousse, lotion, tonic, spray gel or wax.

6. A hair care composition according to claim 1 which is a styling gel, conditioner, leave-in conditioner or hair body-building product.

7. A hair care composition according to claim 1 which also includes a neutralizing agent, conditioning agent or protectant.

8. A hair care composition according to claim 1 wherein (a) is a polyurethane.

9. A hair care composition according to claim 1 wherein (a) is a polyester.

10. A hair care composition according to claim 1 wherein (a) is a polyacrylate.

11. A hair care composition according to claim 1 wherein (a) is a polyolefin.
